(19) United States
(12) Patent Application Publication
(10) Pub. No.: US 2018/0000802 A1
(43) Pub. Date: Jan. 4, 2018

(54) PHARMACEUTICAL COMPOSITION FOR PREVENTION AND TREATMENT OF MENTAL DISEASE WITH ENHANCED NMDAR FUNCTION

(71) Applicants: INSTITUTE FOR BASIC SCIENCE, Daejeon (KR); KOREA ADVANCED INSTITUTE OF SCIENCE AND TECHNOLOGY, Daejeon (KR)

(72) Inventors: Eun-Joon KIM, Daejeon (KR); Su-Yeon CHOI, Daejeon (KR); Wook-Suk CHUNG, Daejeon (KR); Eun-Ee LEE, Daejeon (KR); Ha-Ram PARK, Bucheon-si (KR)

(21) Appl. No.: 15/703,804

(22) Filed: Sep. 13, 2017

Related U.S. Application Data
(62) Division of application No. 14/966,706, filed on Dec. 11, 2015.

(30) Foreign Application Priority Data
Jun. 26, 2015 (KR) 10-2015-0091560

(51) Int. Cl. A61K 31/44; A61K 31/13; A61K 31/439; A61K 31/662

(52) U.S. Cl. CPC A61K 31/44; A61K 31/662; A61K 31/13; A61K 31/439

(57) ABSTRACT

The present invention relates to a pharmaceutical composition for improving a sociability behavior in a patient having a mental disease with an enhanced NMDAR function.

### CROSS-REFERENCE TO RELATED APPLICATION

This application is a divisional of U.S. patent application Ser. No. 14/966,706, filed on Dec. 11, 2015, which claims the benefit of Korean Patent Application No. 10-2015-0091560, filed on Jun. 26, 2015 in the Korean Intellectual Property Office, the entirety of which is incorporated herein by reference.

### BACKGROUND OF THE INVENTION

### 1. Field of the Invention

The present invention relates to a pharmaceutical composition for prevention and treatment of a mental disease with an enhanced N-methyl D-aspartate (NMDA) receptor (“NMDAR”) function.

### 2. Description of the Related Art

One of the most characteristic symptoms shown by patients having mental diseases such as Autism spectrum disorder, Schizophrenia and attention deficit/hyperactivity disorder (ADHD) is a significantly deficient sociability behavior, in which a person takes interest in another person, makes conversations and interacts. Therefore, drugs for improving the sociability behaviors of the patients having mental diseases have been researched (Korean Unexamined Patent 10-2014-0132493 (Nov. 18, 2014)).

However, the causes of Autism spectrum disorder, Schizophrenia, attention deficit/hyperactivity disorder (ADHD), etc. are not completely identified, and the symptoms and conditions of patients vary, so even drugs that improve sociability behavior deficiency of specific patients does not have effects on other patients.

Accordingly, the applicants of the present invention have identified, while researching sociability behavior deficiencies, that sociality can be improved by administrating NMDAR inhibitors in the case of mental diseases with an enhanced NMDAR function.

### SUMMARY OF THE INVENTION

An object of the present invention is to provide a pharmaceutical composition for improving sociability behaviors in a patient having a mental disease with an enhanced NMDAR function.

To achieve the object, the present invention provides a pharmaceutical composition for prevention and treatment of a mental disease with an enhanced NMDAR function, the pharmaceutical composition including an NMDAR inhibitor.

The present invention improves the sociability behavior of a patient having a mental disease with an enhanced NMDAR function.

### BRIEF DESCRIPTION OF THE DRAWINGS

FIG. 1 shows a behavior experiment box used for a 3-chamber sociability test.

FIG. 2 shows an experimental result of wild-type mice and brain-specific angiogenesis inhibitor 1-associated protein 2 (“IRSp53”) deficient mice.

FIGS. 3 and 4 show sociability behavioral changes of mice when memantine and 2-Methyl-6-(phenylethynyl)pyridine (“MPEP”) are administered to the IRSp53 deficient mice, respectively.

FIG. 5 shows the change in NMDAR activation when the memantine and the MPEP are administered to the IRSp53 deficient mice.

FIG. 6 shows the result of synaptic structural and functional analysis of an inner prefrontal cortex of the IRSp53 deficient mice.

FIG. 7 shows the change in nerve cell firing of the inner prefrontal cortex when the memantine is administered to the IRSp53 deficient mice.

### DETAILED DESCRIPTION OF THE INVENTION

The present invention relates to a pharmaceutical composition for prevention and treatment of a mental disease with an enhanced an NMDAR function, the pharmaceutical composition including an NMDAR inhibitor.

In addition, the present invention relates to a pharmaceutical composition for improving sociability behaviors in a patient having a mental disease with an enhanced NMDAR function, the pharmaceutical composition including an NMDAR inhibitor.

Hereinafter, the present invention is described in detail.

NMDAR

N-methyl-D-aspartate (NMDA) receptor (NMDAR) is a specific receptor of a glutamate which is one of main neurotransmitters of an excitatory synapse.

NMDAR Inhibitor

The NMDAR inhibitor signifies medicine suppressing an NMDAR function. The NMDAR inhibitor may be memantine, MPEP, MK-801 (Dizocilpine) or 2-amino-5-phosphonopentanoic acid (“AP5”). The MK-801 is a noncompetitive inhibitor of the NMDAR and binds to an ion binding site to inhibit a receptor function, and AP5 is a selective inhibitor of the NMDAR and binds to a glutamate binding site.

Mental Disease with an Enhanced NMDAR Function

In the present invention, “enhanced NMDAR” signifies that the NMDAR function is more enhanced than a normal person. This may be due to over-expression, enhanced protein functions, etc. The mental disease with the enhanced NMDAR function involves IRSp53 gene damage. In this case, the IRSp53 gene damage signifies that, due to expression suppression, expression disable, mutant, etc. of the IRSp53 gene, a normal level gene expression is difficult.

The mental disease with the enhanced NMDAR function of the present invention has a sociability behavior deficient symptom. The sociability behavior deficiency signifies the generally used sociability behavior deficiency in metal diseases such as autism. In other words, the sociability behavior deficiency includes deficiency of social interactions and social communications, thus indicates difficulty of social interpersonal relations. In addition, the mental disease with the enhanced NMDAR function is any one disease selected from the group consisting of autism, schizophrenia and ADHD.

Patient Having the Mental Disease with the Enhanced NMDAR Function

The patient having the mental disease with the enhanced NMDAR function of the present invention indicates a patient diagnosed with the mental disease with the enhanced NMDAR function. In other words, they are patients having the enhanced NMDAR function among patients having mental diseases of autism, schizophrenia, ADHD, etc.

Pharmaceutical Composition

The pharmaceutical composition of the present invention includes the NMDAR inhibitor. The pharmaceutical composition is for the prevention and treatment of the mental disease with the enhanced NMDAR function or for the improvement of sociability behavior of patients having the mental disease with the enhanced NMDAR function.

The pharmaceutical composition of the present invention targets patients having the enhanced NMDAR function among patients having autism, schizophrenia, attention deficit/hyperactivity disorder, etc. exhibiting sociability behavior deficiency symptoms. In addition, the composition of the present invention targets diseases involving the enhanced NMDAR function among diseases of autism, schizophrenia, attention deficit/hyperactivity disorder, etc. having sociability behavior deficiency symptoms.

The pharmaceutical composition of the present invention may include 0.01 to 99.99 parts by weight of the NMDAR inhibitor based on 100 parts by weight of the composition, and may preferably include 60 to 98 parts by weight. More preferably, the pharmaceutical composition of the present invention may include 90 to 95 parts by weight of the NMDAR inhibitor based on 100 parts by weight of the composition. However, this may be increased or decreased according to the needs of the person to be administrated, and may be adequately increased or decreased according to conditions of dietary life, nutrition state, progression of the disease, amount of sociability behavior deficiency, etc. In addition, the pharmaceutical composition of the present invention may form the NMDAR inhibitor into an appropriate form along with a pharmaceutically allowable carrier. ‘Pharmaceutically allowable’ signifies a composition which does not generally cause allergic reactions such as gastrointestinal disorders, dizziness, etc. or similar reactions when the composition is physiologically allowed and administered to a person.

The pharmaceutical composition of the present invention may be administered orally or parenterally, and may be generally used in a form of a pharmaceutical medicine. A pharmaceutical preparation preferably includes an oral administration preparation such as a tablet, a soft or hard capsule, liquid, a suspension, etc. and the pharmaceutical preparation may be prepared by using the pharmaceutically allowable carrier, for example, in the case of the oral administration preparation, excipients, binders, disintegrants, glydents, solubilizers, suspending agent, preserved agents, extenders, etc.

The administration amount of the pharmaceutical composition of the present invention may be determined by an expert according to various factors such as the state, age, sex, complications, etc. of the patient, and generally, 0.1 mg to 10 g per 1 kg, and more preferably 5 g per 10 mg may be administered for an adult. In addition, 1 day amount or ½, ⅓ or ¼ amount of the 1 day amount of the pharmaceutical composition may be included in a dosage unit, and the pharmaceutical composition may be administered 1 to 6 times a day. However, in the case of a long-term intake for the purpose of health and hygiene or the purpose of adjusting health, the administration amount may be lower than the described range, because the active component is safe, the administration amount may be greater than the described range.

Advantages and features of the present invention, and method for achieving thereof will be apparent with reference to the examples that follow. But, it should be understood that the present invention is not limited to the following embodiments and may be embodied in different ways, and that the embodiments are given to provide complete disclosure of the invention and to provide thorough understanding of the invention to those skilled in the art, and the scope of the invention is limited only by the accompanying claims and equivalents thereof.

<Material and Method>

For the experiment animals of the present invention, wild-type mice backcrossed into a C57BL/6J background and IRSp53 deficient mice were used. The mice were at 2-4 months of age. In addition, in the present invention, all of the experiments were conducted by maintaining a temperature of 20-22° C. and a humidity of 30-40% while the lights are turned on during 7:00 am to 7:00 pm.

The IRSp53 deficient mice were used in the experiment by making the mice from the method disclosed in Kim, M. H. et al. (Enhanced NMDA Receptor-Mediated Synaptic Transmission, Enhanced Long-Term Potentiation, and Impaired Learning and Memory in Mice Lacking IRSp53. J Neurosci 29, 1586-1595, doi:Doi 10.1523/Jneurosci.4306-08.2009). In other words, a particular gene trap cassette was inserted between Exon 3 and Exon 4, which encode the gene sequence expressing the actual protein among the IRSp53 gene (NCBI number: NM_001037755), such that an arbitrarily different sequence is inserted into the original sequence to change the entire sequence, so the gene is not expressed normally, and as a result, the IRSp53 gene expression is suppressed.

As the memantine, which is an NMDAR inhibitor and the MGluR inhibitor, the MPEP indirectly inhibiting the NMDAR is dissolved in physiological saline and used. The memantine is used in a concentration of 10 mg (10 mg/kg) per unit weight, and the MPEP is used in a concentration of 30 mg (30 mg/kg) per unit weight, and the drug was administered to the experiment mice by intraperitoneal injection before 30 minutes of every experiment.

### Experimental Example 1

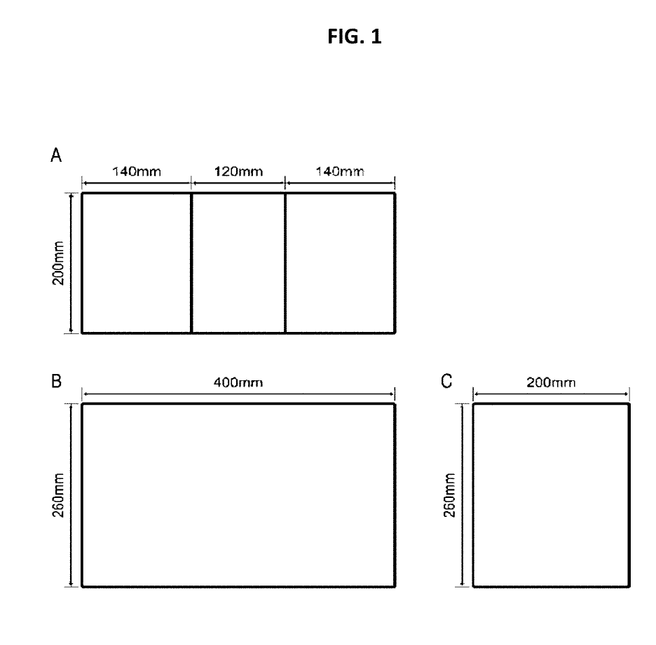

NMDAR was tested with respect to the sociability behavior of hyperactivated mice. The 3-chamber sociability test and the direct interaction test, which are the most widely used among the behavioral experiments for identifying sociability behavior of mice. The 3-chamber sociability test was conducted by using a behavior experiment box separated into 3 sections. FIG. 1 shows a drawing of an experiment box used for the 3-chamber sociability test. A, B, and C of FIG. 1 show the measurement based on the box bottom, the horizontal side surface and the vertical side surface, respectively, and the experiment box was manufactured exposing that the top so the experiment may be photographed by a camera (FIG. 1). In the basic 3-chamber sociability test, a mouse (stranger) was placed in one side and an object (small toy block) without scent was placed in the other side, and the normal mouse (WT) and the deficient mouse were placed in the middle chamber to observe how much attention was given to the other mouse and explored and time was spent.

The detailed experiment method of the present invention is described below. First, the wild-type mouse and the IRSp53 deficient mouse were prepared. In addition, before 10 minutes of the experiment, the mouse to be experimented was put into a soundproof booth in which the experiment was to be conducted. 10 minutes later, the holes in the inner wall were blocked and the mouse was put into the middle area. Another 10 minutes later, the holes were opened, and the mouse was allowed to move freely around the 3 chambers. Then, another mouse (Stranger 1) was put into a cylindrical plastic cup, through which a hole was punctured at a side surface, and the cup was placed upside-down at a corner of one side of the two areas. In the opposite area, a plastic cup, in which objects such as a triangular cone and the like was placed, was placed upside-down. Then, the mice, which are the target of the experiment, were allowed to move around freely for 10 minutes.

Lastly, the object in the cup, in which the object was placed, was removed, and a new mouse (Stranger 2) was put into the cup, and the mouse, which is the target of the experiment, was allowed to move freely for 10 minutes.

In this case, by using the strangers 1 and 2, which are wild-type mice capable of conducting normal growth and sociability behavior, the experiment was designed and conducted such that only the sociability behaviors of the wild-type mice and the IRSp53 gene deficient mice, which are the targets of the experiment, were compared.

The analysis method to obtain the experiment result will be described below.

During 10 minutes of experiment where the Stranger 1 and the object were placed, the time of the mouse staying at the two areas were measured. In addition, to measure the time of the actual mice exploring the stranger 1 or the object, the time when the nose of the mouse crosses over a virtual line 2 cm away from the plastic cup was measured. Based on this number, a preference index showing the sociability behavior was derived. The calculation equation of the preference index is as <Equation 1> to <Equation 3>.

In addition, the same analysis was also conducted for 10 minute experiment performed with the Stranger 1 and Stranger 2.

Preference index (%)={(Time staying in Stranger 1 area−Time staying in the object area)/(Time staying in Stranger 1 area+Time staying in the object area)}×100 <Equation 1>

Preference index (%)={Time of the nose crossing over the virtual line at the cup side of the Stranger 1−Time of the nose crossing over the virtual line at the cup side of the object)/(Time of the nose crossing over the virtual line at the cup side of the Stranger 1+Time of the nose crossing over the virtual line at the cup side of the object)}×100 <Equation 2>

Preference index (%)={(Time staying in Stranger 1 area−Time staying in the Stranger 2 area)/(Time staying in Stranger 1 area+Time staying in the Stranger 2 area)}×100 <Equation 3>

As a result, the time in which the IRSp53 deficient mouse stayed in the room with the mouse was significantly less than the time of the normal mouse, and the time of directly applying the nose to sniff also decreased. In addition, the time of the direct action when noses make contact with each other to sniff and the action of following one another were measured, and compared to the normal mouse, the deficient mouse interacted much less. Therefore, the sociability behavior being seriously deficient in the IRSp53 deficient mouse was observed.

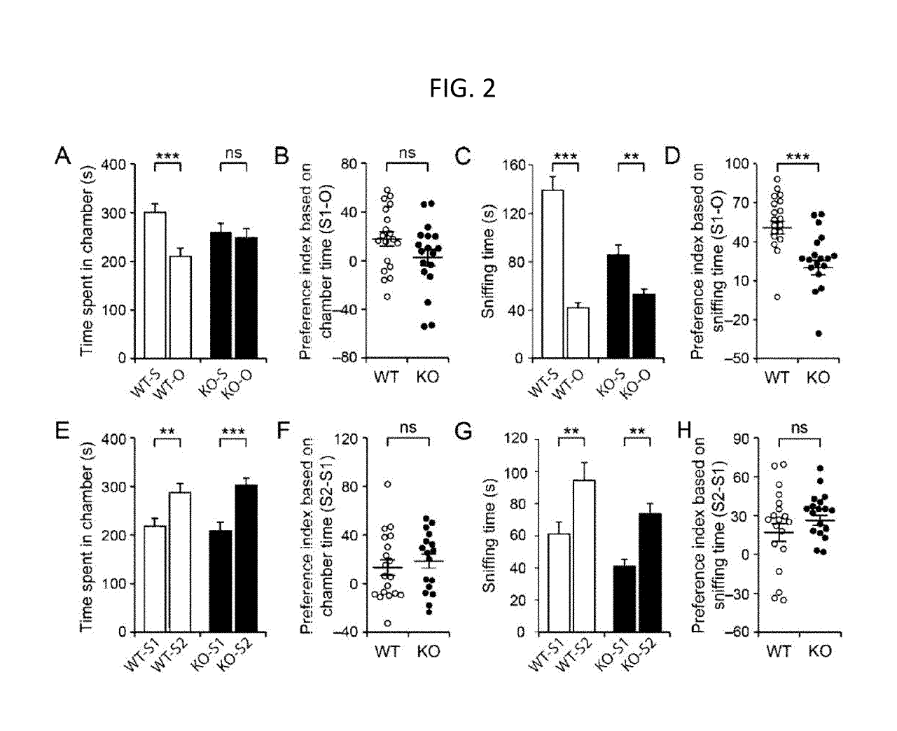

The IRSp53 deficient (KO) mice showed a lower preference for Stranger 1 with respect to the object. This indicates that the IRSp53 deficient mice has a low sociability behavior. Meanwhile, in the experiment having the Stranger 1 and the Stranger 2, the IRSp53 (KO) mouse did not show much difference from the normal mouse, thus, this indicates that the IRSp53 deficient mouse and the other mouse are distinguishable. (A to H of FIG. 2, WT: wild-type mice, KO: IRSp53 deficient mice, S: Stranger, S1: Stranger 1, S2: Stranger 2)

### Experimental Example 2

From the result of the 3-chamber sociability test of Experimental example 1, the IRSp53 deficient mouse was identified as having sociability behavior problems. Therefore, in the present Experimental example 2, whether the sociability behavior deficiency is restored when the NMDAR function in the IRSp53 deficient mice is suppressed was evaluated. The sociability behavior restoring experiment was conducted as described below.

30 minutes before the 3-chamber sociability test, the memantine and the MPEP were intraperitoneally injected to identify the sociability behavior. For reference, the memantine is the direct inhibitor of the NMDAR. In addition, the MPEP is an MGluR inhibitor and indirectly controls the NMDAR through the MGluR which is a glutamate receptor. In the present experiment, the memantine having a concentration of 10 mg/kg was used and the MPEP having a concentration of 30 mg/kg was used. In addition, to eliminate the influence due to the intraperitoneal injection from the effect of the used drug, a saline solution in a same amount of the drug (in other words, the memantine and the MPEP) was intraperitoneally injected into mice as a control group, and the 3-chamber sociability test was conducted as the control group.

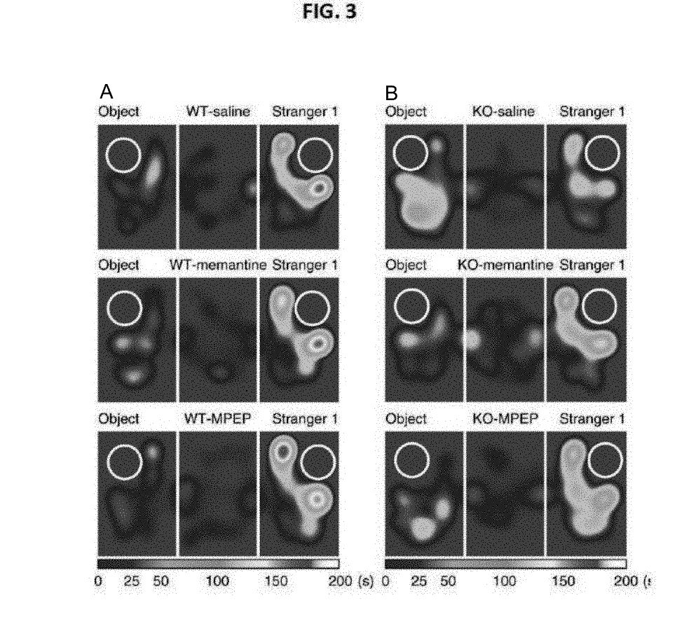

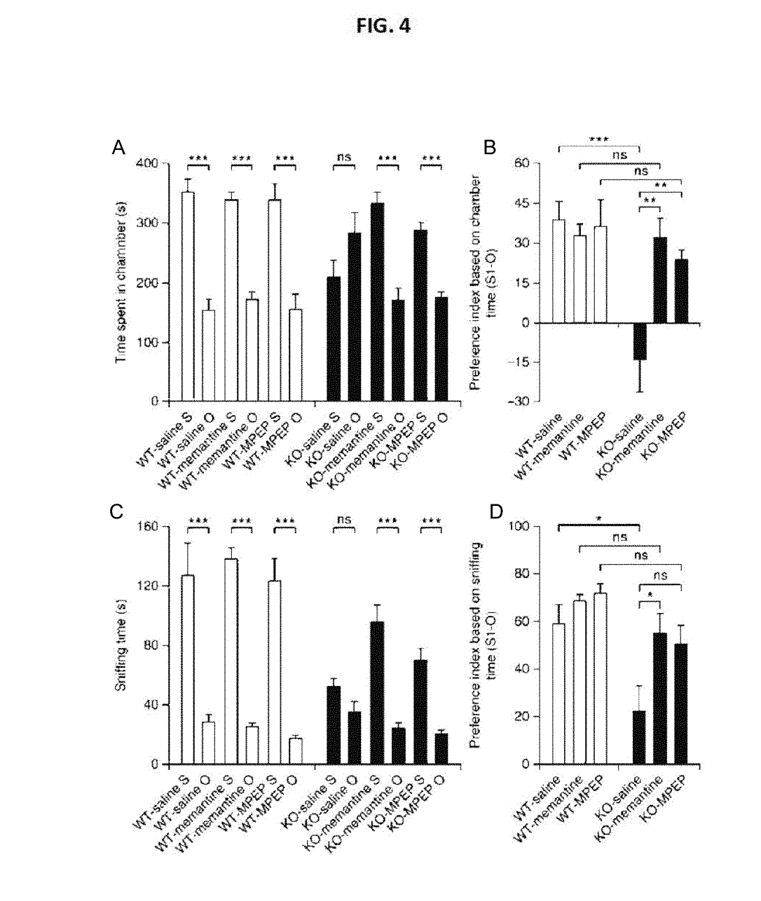

As a result, the wild-type mice injected with the saline solution showed a preference of the normal mice, and a decrease in the mice preference was identified from the IRSp53 deficient mice injected with the saline solution. Meanwhile, the IRSp53 deficient mouse injected with the memantine and the MPEP had a mice preference improved to a level of the wild-type mice and the restoration of the sociability was observed. Therefore, the sociability behavior deficiency symptom shown in the IRSp53 deficient mice restored to the behavior of the normal mice by injecting the memantine and the MPEP was identified. The restoration effect of both drugs was good and there were no sedation due to side effects (A and B of FIG. 3, and A to D of FIG. 4, FIG. 3 shows the movement of the mouse in the 3-chamber sociability test changed to a heat sensing signal, and FIG. 4 shows graphs of the time of the mouse actually staying in the box and the time actually exploring the mouse.)

### <Experimental Example 3> Electrophysiological Measurement of the NMDAR Characteristic of a Nerve Cell of the IRSp53 Deficient Mouse

To observe whether the memantine and the MPEP restore the NMDAR function observed on the hippocampus of the IRSp53 deficient mice to a normal range, the electrical signal flow of the synapses through the NMDAR was measured by the electrophysiological experiment.

<3-1>

When the NMDAR is activated by electrically stimulating the never cell, an ion transferred from the outside of the nerve cell to the inside of the nerve cell through the NMDAR according to the activation level exists, and by the ion concentration difference of the inside and outside of the cell, various proteins inside the cell are also activated, thereby showing electrical reaction of the nerve cell with respect to the stimulation. The state in which this reaction is maintained for 30 minutes or longer is called Long-term potentiation (LTP) and Long-term depression (LTD), and when the NMDAR is intensively activated, the LTP may be induced, and when the NMDAR is weakly activated a long-term weakening, LTD may be induced.

In the present experiment, the Long-term potentiation (LTP) and the Long-term depression experiments were conducted. First, the NMDAR properties of the nerve cell of the IRSp53 deficient mice were electrophysiologically measured. The measurement was experimented and conducted by the method disclosed in the paper of Kim M H et al. (Kim, M. H. et al. Enhanced NMDA Receptor-Mediated Synaptic Transmission, Enhanced Long-Term Potentiation, and Impaired Learning and Memory in Mice Lacking IRSp53. J Neurosci 29, 1586-1595, doi:Doi 10.1523/Jneurosci.4306-08.2009 (2009)). The equipment used for the electrophysiological measurement was MultiClamp 700B amplifier and Axopatch 200B of Molecular Devices, and Clampex 9.2 and Clampfit 9 software of the same company were used. During the experiment, BX50WI microscope of Olympus was used.

In the present experiment, first, for all of the electrophysiological experiment, brain slices were made from the brains of the normal mice and the IRSp53 deficient mice. To make the slices, VT1200S model vibratome of Leica was used. The Long-term potentiation (LTP) and the Long-term depression experiments used mice, which were 21 days to 28 days from birth, and other electrophysiological experiments used mice which were 14 days to 21 days from birth. The brain slices including the hippocampus portions of the mice were made in thicknesses of 300 to 400 μm. During cutting of the slices, the brains were immersed in a cold dissection buffer. The composition of the dissection buffer is as follows: 212 mM of sucrose, 25 mM of NaHCO3, 5 mM of KCl, 1.25 mM of NaH2PO4, 10 mM of D-glucose, 2 mM of sodium pyruvate, 1.2 mM of sodium ascorbate, 3.5 mM of MgCl2 and 0.5 mM of CaCl2. In addition, the dissection buffer was saturated by 95% O2/5% CO2 gas. The cut slices was immersed in an artificial cerebrospinal fluid of 32° C. and moved to room temperature after restoration. The composition of the cerebrospinal fluid is as follows: 125 mM of NaCl, 25 mM of NaHCO3, 2.5 mM of KCl, 1.25 mM of NaH2PO4, 10 mM of D-glucose, 1.3 mM of MgCl2 and 2.5 mM of CaCl2.

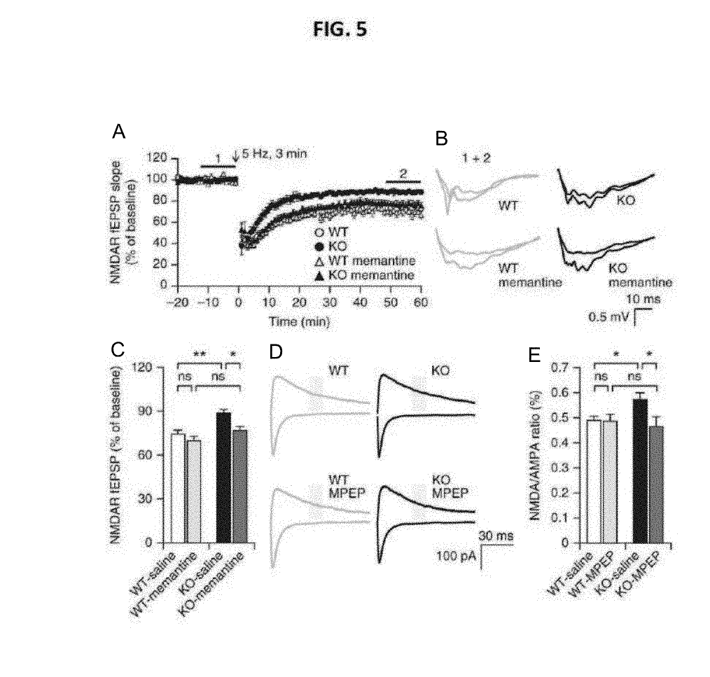

The condition maintained for 30 minutes or more when a reaction occur after the NMDAR is weakly stimulated is called NMDAR-LTD, and in the present experiment, the NMDAR function becomes excessive in the IRSp53 deficient mouse, so when stimulated as same as the wild-type mice, a NMDAR-LTD reaction smaller than the reaction that should originally occur was induced. A weaker NMDAR-LTD is observed in the IRSp53 deficient mouse compared to the normal mouse, and this was restored through the memantine treatment (A to C of FIG. 5, WT: wild-type mice, KO: IRSp53 deficient mice). This indicates that the IRSp53 deficient mice has a problem in removing the NMDA receptor of the nerve cell surface, so excessive NMDA receptors may exist on the surface.

<3-2>

The reactivity occurring when a specific membrane potential state, in which the NMDAR of the nerve cell is fired, is maintained is compared with the reactivity of AMPAR (α-amino-3-hydroxy-5-methyl-4-isoxazolepropionic acid receptor), which is a Glutamate receptor, to evaluate the NMDA/AMRA ratio from which the amount of activation of the NMDAR may be identified.

As a result, in the case of the IRSp53 deficient mouse compared to the wild-type mouse, the NMDAR activation was excessive, so the NMDA/AMPA ratio was increased. When the IRSp53 deficient mouse was treated with the MPEP, the NMDA/AMPA ratio was restored to the level of the wild-type mouse (D and E of FIG. 5, WT: wild-type mice, KO: IRSp53 deficient mice). This result agrees with <3-1> using the memantine.

### Experimental Example 4

The IRSp53 is the fifth most protein existing in the excitatory postsynapse, and is a scaffolding protein directly and indirectly interacting with the neurotransmitter receptor and the signaling protein. The IRSp53 is also known as BAIAP2. The IRSp53 is directly connected to many protein forming actin cytoskeletal, large amount of which exist in the excitatory synapse and is required for specifically maintaining the synaptic structure.

The brain slices including the hippocampus from each of the wild-type mouse and the IRSp53 deficient mouse were prepared, and the electrophysiological equipment was used to induce a reaction stimulating specifically the NMDAR directly on each never cell.

As a result, in the wild-type mouse, the NMDAR function was activated normally, thus the reaction was observed, whereas, in the IRSp53 deficient mouse, about a 20% increased reaction compared to the wild-type mouse was observed (E of FIG. 5, comparison of WT-Saline/KO-Saline NMDA/AMPA ratio). Therefore, in the IRSp53 deficient mouse, the NMDAR function was abnormally increased.

### Experimental Example 5

<5-1>

In general, the medial prefrontal cortex (mPFC) is known to be important as the brain region responsible for the sociability behavior. A chemical product for fluorescence was filled in the nerve cell of the medial prefrontal cortex of the wild-type mouse and the IRSp53 deficient mouse and the structure and the form of the nerve cell were observed by a fluorescence microscope.

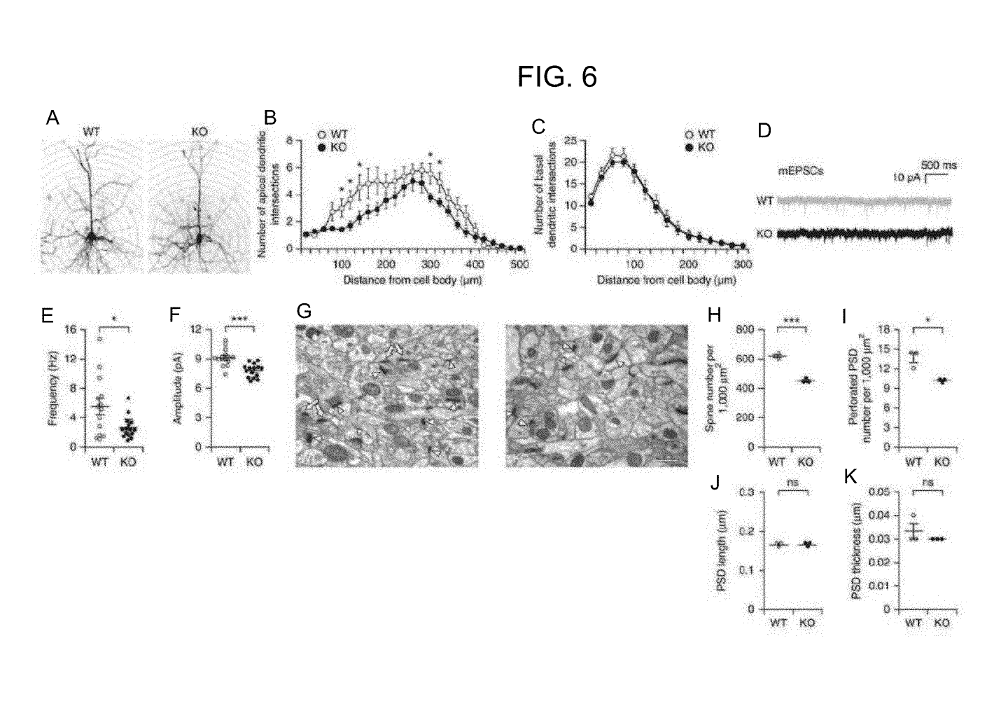

In the nerve cell structure, when comparing the structure of dendrite having the role of receiving the stimulation and information from the brain, the development of the dendrite in the IRSp53 deficient mouse was lower than in the wild-type mouse (A and C of FIG. 6). Meanwhile, when observing the dendritic spine of the nerve cell by the electrophysiological experiment, the IRSp53 deficient mouse had a lower development of the dendritic spine of the nerve cell (D to F of FIG. 6). In addition, when the electrical reaction occurring by a voluntary secretion of the glutamate without any stimulation to the nerve cell is measured, the number of the dendrite reacting to the glutamate may be inferred, based on this result and from observing the dendritic spine structure of the nerve cell of the medial prefrontal cortex through an actual electron microscope, the number of the dendrite decreased in the IRSp53 deficient mice (G, H, I, J and K of FIG. 6).

Therefore, in the case of the IRSp53 deficient mouse, the excitatory synapse structure was impaired, and the synaptic electrical signal and the nerve cell firing were abnormal (A to K of FIG. 6).

<5-2>

The memantine was administered to the IRSp53 deficient mouse, and the synapse of the medial prefrontal cortex was structurally and functionally analyzed. In the experiment, the brain slices were not made, an anesthetic drug (1.5 g/kg) named Urethane was used on the mouse to perform the electrophysiological experiment in a condition in which the neural circuitry in the mouse is maintained, and the electrical signal firing of the nerve cell was observed,

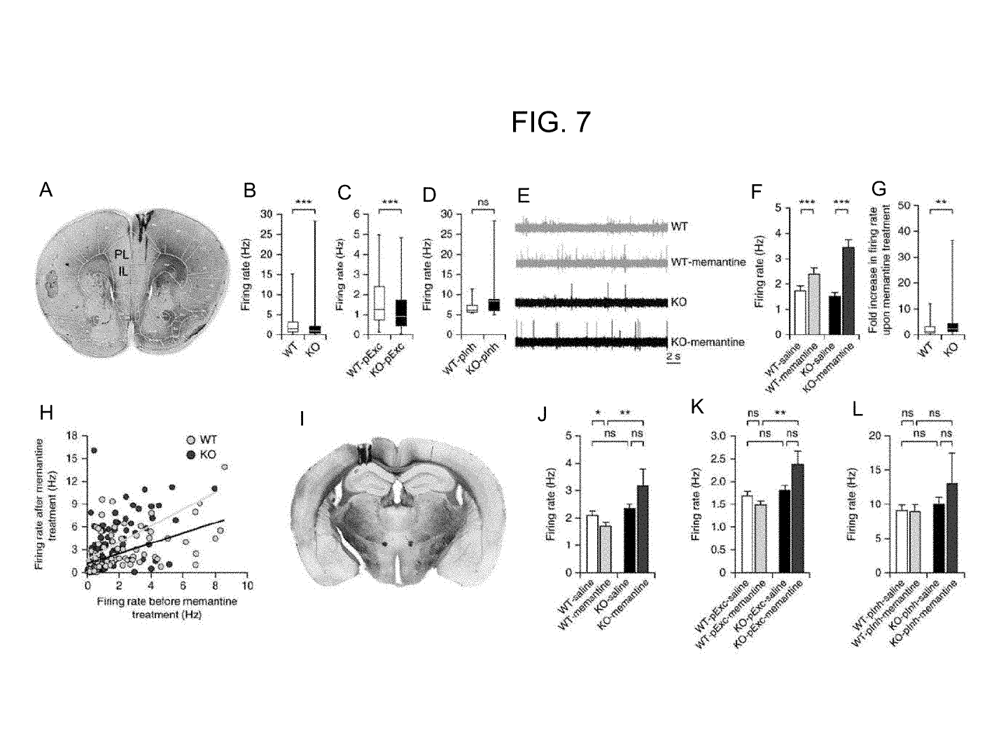

In detail, in the experiment, the mouse was anesthetized, a glass electrode for measuring the electrical signal was inserted in the medial prefrontal cortex and the hippocampus, and measured. A and I of FIG. 7 show the positions through which each electrode is inserted. From the result of comparing the nerve cell firing of the medial prefrontal cortex of the IRSp53 deficient mouse, in the IRSp53 deficient mouse, the nerve cell firing of the medial prefrontal cortex decreased. Specifically, in the IRSp53 deficient mouse, the nerve cell firing specific to the excitatory nerve cell decreased (B, C and D of FIG. 7).

The memantine was intraperitoneally injected (10 mg/kg) into the IRSp53 deficient mice such that the nerve cell firing may be induced after 30 minutes, and from the observation, the reactivity with respect to the IRSp53 deficient mouse was high and the drug showed good effects.

Meanwhile, in the case of the hippocampus, from the result of injecting the memantine into the wild-type mouse and the IRSp53 deficient mouse and observing the nerve cell firing change, the firing was greater and the showed better effect in the IRSp53 deficient mouse compared to the wild-type mice (J, K and L of FIG. 7).

Therefore, when the IRSp53 deficient mouse was treated with the memantine, the nerve cell firing of the medial prefrontal cortex restored to the normal range (A to L of FIG. 7).

What is claimed is:

1. A method for treating a mental disease having enhanced N-methyl-D-aspartate (NMDA) receptor (NMDAR) function in a patient having the mental disease, the method comprising:
administering an NMDAR inhibitor composition to the patient.

2. The method of claim 1, wherein the NMDAR inhibitor composition is selected from memantine, MPEP, MK-810, or AP5.

3. The method of claim 1, wherein the mental disease comprises a sociability behavior deficiency symptom.

4. The method claim 1, wherein the patient has a deficient IRSp53 gene.

5. The method of claim 4, wherein the deficient IRSp53 gene does not express the IRSp53 protein.

6. The method of claim 1, wherein the mental disease is selected from the group consisting of autism, schizophrenia, and attention deficit/hyperactivity disorder (ADHD).

7. The method of claim 1, wherein the NMDAR inhibitor composition comprises memantine.

8. The method of claim 1, wherein the NMDAR inhibitor composition comprises MPEP.

9. A method for decreasing enhanced N-methyl-D-aspartate (NMDA) receptor (NMDAR) function in a patient having enhanced NMDAR function, the method comprising:
administering an NMDAR inhibitor composition to the patient.

10. The method of claim 9, wherein the NMDAR inhibitor composition is selected from memantine, MPEP, MK-810, or AP5.

11. The method of claim 9, wherein the patient has a deficient IRSp53 gene.

12. The method of claim 9, wherein the mental disease is selected from the group consisting of autism, schizophrenia, and attention deficit/hyperactivity disorder (ADHD).

13. A method for treating schizophrenia or attention deficit/hyperactivity disorder (ADHD) in a patient having schizophrenia or attention deficit/hyperactivity disorder (ADHD) and enhanced N-methyl-D-aspartate (NMDA) receptor (NMDAR) function, comprising:
administering an NMDAR inhibitor composition to the patient.

14. The method of claim 13, wherein the patient has a sociability behavior deficiency symptom.

15. The method of claim 14, wherein the sociability behavior deficiency symptom includes social interaction deficiencies and social communication deficiencies.

16. The method of claim 13, wherein the patient has a deficient IRSp53 gene.

17. The method of claim 16, wherein the deficient IRSp53 gene does not express the IRSp53 protein.

18. The method of claim 13, wherein the NMDAR inhibitor composition comprises memantine, MPEP, MK-810, or AP5.

19. The method of claim 13, wherein the NMDAR inhibitor composition is memantine.

20. The method of claim 13, wherein the NMDAR inhibitor composition is MPEP.
